(19) United States
(12) Patent Application Publication
(10) Pub. No.: US 2017/0340544 A1
(43) Pub. Date: Nov. 30, 2017

(54) COSMETIC COMPOSITION FOR IMPROVING HEALTH OF SCALP AND METHOD OF PREPARING THE SAME

(71) Applicant: OLIPASS Cosmeceuticals Company, Yongin-si (KR)

(72) Inventors: Shin CHUNG, Yongin-si (KR); Jongho PARK, Seoul (KR); Su Jung KIM, Seoul (KR)

(21) Appl. No.: 15/601,496

(22) Filed: May 22, 2017

(30) Foreign Application Priority Data
May 31, 2016 (KR) 10-2016-0067375

(51) Int. Cl. A61K 8/64; A61Q 5/00; A61K 8/60; A61Q 7/00; A61K 8/34

(52) U.S. Cl. CPC A61K 8/64; A61Q 7/00; A61K 8/345; A61K 8/60; A61Q 5/00

(57) ABSTRACT

This invention relates to a cosmetic composition, which controls sebum secretion of the scalp to thus create an optimal environment for hair growth and activates growth factors for promoting hair strengthening and growth, thereby improving the health of the scalp. A method of preparing the same is also provided. In the cosmetic composition, an oligopeptide functions to create an environment suitable for hair growth by controlling the sebum secretion of the scalp, and also to activate growth factors that are effective at preventing hair loss by promoting hair growth through improvement of blood circulation in the scalp and by strengthening hair through hair follicle activity, thereby improving the health of the scalp and preventing hair loss. The oligopeptide of the invention can exhibit high safety and stability of use and high absorbability into the skin and is thus excellent in improving the health of the scalp, compared to the use of existing peptide compositions.

### CROSS-REFERENCE TO RELATED APPLICATIONS

This application claims the priority of the Korean Patent Application NO 10-2016-0067375 filed on May 31, 2016, in the Korean Intellectual Property Office, the disclosure of which is incorporated herein by reference.

### BACKGROUND OF THE INVENTION

### 1. Technical Field

The present invention relates to a cosmetic composition for improving scalp health and a method of preparing the same, in which the cosmetic composition controls sebum secretion of the scalp to thus create an optimal environment for hair growth and also activates growth factors able to promote hair strengthening and growth, thereby improving the health of the scalp.

### 2. Description of the Related Art

The scalp is a skin tissue that covers the surface of the skull, and is a common site on which the hair grows and which buffers external physical stimuli or chemical changes to thus protect the inside thereof. The scalp is rich in hair follicles and blood vessels and has a very dense nerve distribution compared to other skin tissues, and also provides an environment in which the blood vessels and lymph vessels in the subcutaneous tissue supply nutrients and oxygen into the scalp to induce hair growth.

The scalp enables harmful substances and waste materials to be excreted from the body. When keratin and waste materials accumulate together with the sebum on the surface of the scalp, the pores of the scalp may become clogged, undesirably causing inflammation and thinning the hair. Furthermore, the scalp is covered with hair, unlike other skin, and thus has high humidity and is weakly resistant to microbial proliferation.

A healthy scalp is characterized in that it secrets an appropriate amount of sebum and is elastic and the scalp color is clear, transparent and shiny. When problems occur on such a scalp, a dry scalp, an oily scalp, a sensitive scalp, a dandruff scalp, or an alopecia scalp may result, all of which are referred to as problematic scalps. The types of problematic scalp are specified below.

In the case of a dry scalp, aged keratin is thick and the scalp appears turbid due to abnormal keratinization. Because of an oil-water imbalance, the scalp is very dry and crumbly and has no gloss. Furthermore, blood circulation in the scalp is not efficient and thus dandruff may build up and hair loss may be caused due to the abnormal keratinization.

In the case of an oily scalp, dandruff, keratin and sebum are entangled together on the scalp due to excessive sebum secretion, and the secreted sebum may clog the pores of the skin, and thus inflammation may occur or hair growth may be disturbed, undesirably causing hair loss.

In the case of a sensitive scalp, capillary vessels are enlarged, whereby the scalp responds sensitively due to a fever phenomenon or reacts painfully even to weak external stimuli. Erythema, inflammation and thin capillaries are visible on the surface of the scalp and the hair becomes very thin.

In the case of a dandruff scalp, dandruff may accumulate due to abnormal proliferation of a dandruff-causing virus and is thus accompanied by itching, and is classified into dry dandruff and oily dandruff. Dry dandruff results from a dry scalp and is in a scaly form, and oily dandruff is characterized in that the sebum is entangled with keratin.

As for an alopecia scalp, hair falls out before the completion of the lifespan thereof. When hair loss begins, there are few cases where hair is lost all at once, and the hair gradually becomes thin and is then lost, and such a scalp is characterized by a red tone.

In particular, hair loss has been regarded as a worry those over age 50, but there has recently been an increase in hair loss among young people in their 20s and 30s. In this regard, people who are worried about hair loss number about 6 million to 7 million, among which the number of women is more than 3 million, showing that the female population suffering from alopecia is increasing rapidly.

Typical causes of hair loss may include stress, excessive use of shampoo or hair mousse, hair and scalp damage due to perms or dyeing and use of a hair dryer, imbalance of dandruff or sebum secretion, hyperactivity of male hormones, excessive sebum secretion, poor blood circulation, deterioration of functions of the scalp due to peroxides and microbes, high-fever illness, use of anticancer drugs and various anti-thyroid drugs, administration of oral contraceptives, poor nutrition, ingestion of processed foods, and genetic factors.

There are some differences in hair loss between men and women. For male alopecia, more than 95% of cases are attributed to the excessive secretion of male hormones based on genetic predisposition. Specifically, testosterone, which is a male hormone secreted from the body, is converted into DHT (Dihydrotestosterone) by a specific enzyme. When testosterone comes into contact with hair follicle dermal papilla cells, it is activated by the action of an enzyme, and is thus converted into the active male hormone DHT. As such, the concentration of DHT in the body becomes relatively high, and thus hair follicles shrink, whereby hair loss quickly progresses.

On the other hand, DHT is also a major cause of female alopecia but fortunately, the destruction of hair follicles by DHT is suppressed through the secretion of the female hormone.

With regard to the treatment of such a hair loss phenomenon, various methods such as topical application, oral administration, use of health supplements, gene transplantation, hair transplantation and alternative medicine have been performed, but it is known that there are no treatment methods or substances that show satisfactory results yet. In order to prevent such hair loss and improve the health of the scalp, a cosmetic composition for use in hair-loss prevention or hair growth is provided. For conventionally commercially available products, chemicals are mostly used as the major ingredient, undesirably causing side effects on the scalp. Furthermore, when Minoxidil, approved by the FDA, is employed as a hair loss drug, unexpected side effects, such as decreased sexual function due to imbalance of homeostasis, allergies, depression, and local inflammation, may occur and thus the application thereof entails some risk.

As described above, the causes of hair loss are various. In the case where nutrients that may constitute the hair are not properly supplied due to blood circulation disorders on account of capillary lesion or compression or where the scalp cells become abnormal due to excessive accumulation of physiological substances and impairment of osmotic homeostasis, there are proposed many causes, such as abnormal cell death attributed to abnormality of the hair follicle cycle, keratinization attributed to excessive sebum secretion, a worsened scalp environment attributed to waste materials, degradation of scalp functions by microbes, etc., resulting in hair loss. Hence, hair loss is not relieved even though only a single problem is solved. Accordingly, a composite that is able to solve various causes at once and to enhance synergistic effects for relieving hair loss and improving the health of the scalp is required.

Korean Patent Application Publication No. 2016-0054087 (Laid-open date: May 16, 2016) discloses caffeoyl pentapeptide for alleviating skin irritation, preventing hair loss and promoting hair growth and a cosmetic composition for the prevention of hair loss and the promotion of hair growth containing the same. Specifically, caffeic acid and pentapeptide are linked to each other through chemical bonding, thereby providing caffeoyl pentapeptide for alleviating skin irritation, preventing hair loss and promoting hair growth and a cosmetic composition containing the same to thereby exhibit the effects of prevention of hair loss and promotion of hair growth.

Korean Patent No. 1541533 (Registration Date: Aug. 28, 2015) discloses a cosmetic composition for preventing hair loss, promoting hair growth, inhibiting the production of dandruff, and relieving scalp inflammation, which contains, as an active ingredient, a composite composition comprising a Polygonum multiflorum root extract, a Sophora flavescens root extract, a Glycyrrhiza glabra root extract, IGF-1 (Insulin-like Growth Factor-1), PDGF (Platelet-derived growth factor) and MSM (Methyl Sulfonyl Methane).

In such conventional techniques, however, the protein ingredients, namely peptide and/or growth factor, contained in the cosmetic composition have difficulty penetrating into the skin, making it difficult to obtain satisfactory effects for the prevention of hair loss or the improvement of the health of the scalp upon real-world application.

### CITATION LIST

### Patent Literature

(Patent Document 1) Korean Patent Application Publication No. 2016-0054087 (Laid-open date: May 16, 2016)

(Patent Document 2) Korean Patent No. 1541533 (Registration Date: Aug. 28, 2015)

### SUMMARY OF THE INVENTION

Accordingly, the present invention is intended to provide a cosmetic composition, which is effective at preventing hair loss through improvement in the health of the scalp by activating growth factors for growing and strengthening hair, as well as by creating an optimal environment for hair growth through appropriate sebum secretion, in order to prevent hair loss from occurring because of various scalp problems including deterioration of the scalp environment due to waste and keratin formed by excessive sebum secretion, and a difficulty of supplying nutrients to the hair due to blood circulation disorders.

Therefore, the present invention provides a cosmetic composition for improving the health of a scalp, comprising: an insulin-like growth factor-1, thymosin-β4 and an oligopeptide, wherein the oligopeptide is configured to include glycine as an N-terminus amino acid and to have a length of 10 to 15 amino acids.

The cosmetic composition may comprise 100 parts by weight of the insulin-like growth factor-1, 17 to 67 parts by weight of the thymosin-β4 and 8 to 25 parts by weight of the oligopeptide, and the insulin-like growth factor-1, the thymosin-β4 and the oligopeptide may be incorporated in a carrier and stabilized.

The carrier may include a macromolecule, a microassembly, a microparticle, a microsphere, a nanosphere, a liposome, an emulsion or a combination thereof.

The cosmetic composition preferably further comprises a complex buffer solution including hexose, a polyhydric alcohol and an electrolyte compound to maintain the homeostasis of an acid and a base inside and outside scalp cells. Here, the complex buffer solution may include 7 to 28 wt % of the hexose, 2 to 8 wt % of the polyhydric alcohol, 0.23 to 1.559 wt % of the electrolyte compound and the remainder of water, and the sum of the hexose, the polyhydric alcohol, the electrolyte and the water does not exceed 100 wt %.

The cosmetic composition may be provided in any formulation selected from the group consisting of a solution, a suspension, an emulsion, a paste, a gel, a cream, a lotion, a powder, an oil, a soap, a cleansing foam, a shampoo, a rinse, a treatment, a wax, and a spray.

In addition, the present invention provides a method of preparing a cosmetic composition for improving the health of a scalp, comprising: preparing an oligopeptide configured to include glycine as an N-terminus amino acid and to have a length of 10 to 15 amino acids; mixing the oligopeptide with an insulin-like growth factor-1 and thymosin-β4, thus preparing a mixture; and adding the mixture with any additive selected from the group consisting of an oil, a surfactant, a moisturizer, a conditioning agent, an antioxidant, a thickener, a binder, a pH controller, a buffering agent, a colorant and a fragrance, thus obtaining the cosmetic composition.

In the mixing, the oligopeptide, the insulin-like growth factor-1 and the thymosin-β4 are preferably included in a solvent obtained by mixing an organic phase, a water phase and a surfactant, homogenized at a high pressure and then cooled, and more preferably, a complex buffer solution including hexose, a polyhydric alcohol and an electrolyte compound is further included.

According to the present invention, an oligopeptide can control sebum secretion of the scalp to thus create an environment suitable for hair growth, and can activate growth factors having an effect of preventing hair loss by promoting hair growth through improvement of blood circulation in the scalp and by strengthening hair through hair follicle activity, ultimately improving the health of the scalp and thus preventing hair loss.

In particular, the oligopeptide of the present invention is increased not only in safety and stability of use but also in absorbability into the skin compared to existing peptide compositions, and is thus excellent in improving the health of the scalp.

Furthermore, an insulin-like growth factor-1, thymosin-β4 and an oligopeptide are incorporated in a carrier and thus stabilized, whereby the resulting composition causes no irritation on the scalp and penetrates deeply at a high concentration, thus improving the health of the scalp and preventing hair loss.

### DESCRIPTION OF SPECIFIC EMBODIMENTS

Before preferred embodiments of the present invention are described in more detail, it must be noted that the terms and words used in the present specification and claims should not be interpreted as being limited to typical meanings or dictionary definitions, but should be interpreted as having meanings and concepts relevant to the technical scope of the present invention.

As used herein, when any part “includes” any element, this means that another element is not excluded, but may be further included unless otherwise specifically mentioned.

As used herein, the terms “first”, “second”, etc. may be used to distinguish one element from another element, and the scope of the invention should not be limited by these terms. For example, the first element may be referred to as a second element or the second element may be referred to as a first element.

The reference numerals in individual steps are used for the sake of description, and do not explain the order of the steps. Individual steps may be performed differently from the described order unless the specific order is explicitly stated in context. That is, individual steps may be performed in the described order or in the reverse order, or may be substantially simultaneously conducted.

As used herein, the term “hair” generally refers to hair growing on all areas of the body, the term “hair growth” means hair growing from the skin, and the term “hair loss” may mean that there is no hair on the area where hair normally exists, for example that the terminal hair of the scalp is missing. Furthermore, “prevention of hair loss” may mean that hair loss from hair follicles or the scalp is inhibited or decreased.

Hereinafter, a detailed description will be given of a cosmetic composition for improving the health of the scalp and a method of preparing the same according to the present invention.

The cosmetic composition for improving the health of the scalp according to the present invention may include an insulin-like growth factor-1, thymosin-β4, and an oligopeptide.

The oligopeptide may have a length of 10 to 15 amino acids, in which an N-terminus amino acid is glycine, and may be configured such that 9 to 14 amino acids selected from among alanine (Ala), valine (Val), leucine (Leu), isoleucine (Ile), threonine (Thr), serine (Ser), cysteine (Cys), methionine (Met), aspartic acid (Asp), asparagine (Asn), glutamic acid (Glu), glutamine (Gln), lysine (Lys), arginine (Arg), histidine (His), phenylalanine (Phe), tyrosine (Tyr), tryptophan (Trp) and proline (Pro) are connected in the preferred order to glycine, which is the N-terminus amino acid.

For example, the oligopeptide may include Gly-Gln-Gln-Lys-Arg-Ser-Asp-Arg-Leu-Asn-Leu-Ser-Arg, Gly-His-Lys-Gly-Gin-Leu-Tyr-Val-Gln-Len, Gly-Gln-Gln-Lys-Asp-Val-Tyr-Val-Gln-Leu-Tyr, Gly-Glu-Gln-Lys-Asp-Val-Tyr-Val-Gln-Leu-Tyr, Gly-Gln-Lys-Gly-His-Lys-Asp-Val-Leu-Tyr, Gly-His-Lys-Lys-Lys-Gly-His-Lys-Gly-His, Gly-His-Lys-Lys-Gly-His-Lys-Lys-Lys-Gly-His-Lys-Asp-Val, Gly-His-Lys-Gly-His-Lys-Asp-Val-Gln-Leu-Tyr, Gly-His-Lys-Lys-Gly-His-Lys-Glu-Gln-Arg-Tyr- Val-Gln-Leu-Tyr or Gly-His-Lys-Gly-His-Lys-Lys-Lys-Gly-His-Lys, and may be a peptide in which the selected amino acids are sequentially connected in the predetermined order.

Thus, the oligopeptide contained in the cosmetic composition of the present invention is not particularly limited, so long as it is configured such that the N-terminus amino acid is glycine (Gly) and 9 to 14 predetermined amino acids are sequentially connected to Gly.

It is preferred that the oligopeptide is appropriately selected depending on the aspect and method of using the cosmetic composition of the present invention and the status of health of users.

More preferably, the oligopeptide is provided in the form of a salt, which is made during the final separation and purification of the compound or by the reaction between an amino group and an appropriate acid, and examples of the acid addition salt may include, but are not limited to, acetate, adipate, alginate, citrate, aspartate, benzoate, benzenesulfonate, bisulfate, butyrate, camphorate, camphor sulfonate, digluconate, glycerophosphate, hemisulfate, heptanoate, hexanoate, formate, fumarate, hydrochloride, hydrobromide, hydroiodide, 2-hydroxyethanesulfonate, lactate, maleate, mesitylene sulfonate, methane sulfonate, naphthylene sulfonate, nicotinate, 2-naphthalene sulfonate, oxalate, pamoate, pectinate, persulfate, 3-phenylpropionate, picrate, pivalate, propionate, succinate, tartrate, trichloroacetate, trifluoroacetate, phosphate, glutamate, bicarbonate, para-toluenesulfonate, and undecanoate.

Also, examples of an acid useful for forming the above acid addition salt may include, but are not limited to, inorganic acids such as hydrochloric acid, hydrobromic acid, sulfuric acid and phosphoric acid, and organic acids such as oxalic acid, maleic acid, succinic acid and citric acid.

Preferably, the oligopeptide of the present invention is provided in the form of a trichloroacetate salt or an acetate salt.

Also, the insulin-like growth factor-1 and thymosin-β4, which are contained in the cosmetic composition for improving the health of the scalp according to the present invention, function to prevent hair loss by promoting hair growth through the improvement of scalp circulation or by strengthening hair through hair follicle activity, thereby affecting hair growth in the skin tissue.

The insulin-like growth factor-1, which is known to form bones, muscles, nerves, blood vessels and tissues, regenerate damaged cells and inhibit cell death, acts as a growth hormone involved in tissue formation to thus help in forming new blood vessels, and is able to favorably control the improvement of cell regeneration ability, proliferation of keratinocytes and growth of hair follicles, whereby the growth of the hair is maintained, thus preventing hair loss. Also, thymosin-β4 was discovered in the thymus of cattle in 1981, and is known to be a protein having weak acidity configured such that 43 amino acids are linked to each other, with a molecular weight of 4,982 Da, the theoretical isoelectric point of which is 5.1. Furthermore, there are many polar amino acids such as glutamic acid and lysine residues, 11 negatively charged amino acids including aspartic acid and glutamic acid, and 9 amino acids including lysine and arginine residues, and the virtual secondary structure of thymosin-β4 has two helix structures.

In this way, thymosin-β4 plays an important role in modulating cell migration and differentiation and is able to help in wound healing and angiogenesis. By increasing the size and distribution of hair vessels, the sizes of the hair follicles and hair shafts may be improved, thus promoting hair growth.

The cosmetic composition for improving the health of the scalp according to the present invention may comprise 100 parts by weight of the insulin-like growth factor-1, 17 to 67 parts by weight of thymosin-β4, and 8 to 25 parts by weight of the oligopeptide. Given the above amount ranges obtained through repeated experiments, the oligopeptide is able to activate the growth factors, thereby growing hair through improvement of blood circulation in the scalp and strengthening hair through hair follicle activity, and also, sebum secretion is controlled to thus create an environment optimal for hair growth. If the amounts thereof fall out of the above ranges, the effects of preventing hair loss and improving hair growth and the health of the scalp cannot be obtained, or there are no obvious effects even upon excessive use thereof.

In order to improve the health of the scalp to thus promote hair growth and to prevent hair loss, the cosmetic composition for improving the health of the scalp according to the present invention, including the insulin-like growth factor-1 and thymosin-β4, may further include any growth factor appropriately selected from the group consisting of an epidermal growth factor (EGF), a transforming growth factor-α (TGF-α), a transforming growth factor-β (TGF-β), a fibroblast growth factor-2 (FGF-2), a keratinocyte growth factor (KGF), a stem cell factor (SCF), a platelet-derived growth factor (PDGF), a vascular endothelial growth factor (VEGF), and a basic fibroblast growth factor (bFGF), as necessary. The oligopeptide of the present invention activates such growth factors, thereby forming and maintaining the optimal scalp state for hair growth.

Preferably useful are a keratinocyte growth factor (KGF), a vascular endothelial growth factor (VEGF), and a basic fibroblast growth factor (bFGF). Here, KGF is associated with the growth and differentiation of keratinocytes, controls new hair production, functions as a modulator important for epithelial-mesenchymal interaction, and protects keratinocytes from UV light, thus preventing cell damage and hair loss. VEGF increases the plasma protein permeability of capillaries and thus promotes not only cell division and migration but also angiogenesis, and furthermore, it is involved in maintaining blood vessels and increases the size and distribution of hair vessels, whereby the sizes of hair follicles and hair shafts are improved and hair growth is promoted. The basic fibroblast growth factor functions to increase the synthesis of collagen and elastin to thus promote hair growth. More preferably, based on 100 parts by weight of the insulin-like growth factor-1, 70 to 100 parts by weight of the keratinocyte growth factor, 80 to 120 parts by weight of the vascular endothelial growth factor, and 70 to 130 parts by weight of the basic fibroblast growth factor may be further included.

When the insulin-like growth factor-1, thymosin-β4 and oligopeptide, which are incorporated in a carrier and thus stabilized, are prepared into a cosmetic composition using an emulsion base, the effects of the present invention may be maximized. Preferably, the insulin-like growth factor-1, thymosin-β4 and oligopeptide are incorporated in a carrier such as a macromolecule, a microassembly, a microparticle, a microsphere, a nanosphere, a liposome, an emulsion or a combination thereof, and are thus stabilized, thereby exhibiting the effect of moisturizing the scalp due to the emulsion base, ultimately increasing the rate of absorption into the scalp, resulting in improved effects.

The cosmetic composition for improving the health of the scalp according to the present invention may further include a buffer solution, which controls the acid-base balance inside and outside the scalp cells to thus effectively penetrate a monosaccharide serving as the energy source of cells, and also promotes the secretion of a neurotransmitter to modulate the blood flow in the dermis in the scalp so as to aid in improving the health of the scalp.

The electrolyte compound is typically used for cosmetic compositions and may be used without particular limitation so long as it is able to supply inorganic materials deeply into the scalp and does not cause allergic reactions or irritation when coming into contact with the skin. Preferably, at least one selected from among sodium chloride, potassium chloride, calcium chloride and sodium sulfate is used, and more preferably 0.1 to 1 wt % of sodium chloride, 0.01 to 0.055 wt % of potassium chloride, 0.01 to 0.034 wt % of calcium chloride and 0.11 to 0.47 wt % of sodium sulfate are used.

Sodium chloride contained in the electrolyte compound is used in an amount of 0.1 wt % or less so as to be equal to the osmotic pressure of the cells. If the amount thereof exceeds 1 wt %, the acid-base balance inside and outside the cells may break down due to the osmotic pressure difference, thus aggravating the health of the scalp. If the amount of sodium ions produced through ionization of sodium chloride is less than 0.1 wt %, hexose and polyhydric alcohol, which are contained in the complex buffer solution and may thus be used as a nutrient in the cells, may be decreased in the efficiency of secondary active transport within the cells, and thus nutrients may not be supplied properly.

The potassium ions ionized from potassium chloride contained in the electrolyte compound are present in the cells, and maintain an electrical equilibrium relation with sodium ions having the highest concentration among cations contained in the extracellular fluid and may thus play an important role in the active transport of materials. The calcium ions may produce diacylglycerol through decomposition of the inositol phospholipid of the cell membrane in the extracellular fluid to thus activate the protein kinase and cause the proliferation of keratinocytes. Furthermore, sodium sulfate is metabolized in the body to generate sodium bicarbonate, thus causing a buffering effect, thereby maintaining a homeostatic acid-base equilibrium.

Accordingly, the inorganic material generated from the electrolyte compound is contained in the body, thus maintaining homeostasis of the body. Hence, when it is included as an isotonic solution in the body, no allergic reaction occurs on the surface of the scalp or within the epidermis, and the acid-base equilibrium is maintained, thus facilitating material transport of the cell membrane. As well, not only hexose and polyhydric alcohol but also oxygen, proteins, lipids and other saccharides may be efficiently supplied, thereby improving the health of the scalp and preventing hair loss.

Hexose is a monosaccharide having 6 carbon atoms, and may include at least one selected from the group consisting of D-glucose, D-gulose, D-mannose, D-galactose, D-idose, D-talose, D-allose, D-altrose, D-fructose, D-tagatose, and L-sorbose. Preferably useful is a mixture of D-glucose and D-fructose.

Due to the migration of D-fructose into the cells from the extracellular fluid layer, D-fructose is easily used as an energy source for scalp cells, and facilitates the passage of D-glucose through the cells owing to at least a 2-fold difference in osmotic pressure. As it is provided as an intracellular nutrient source, hair growth may be improved. Furthermore, the growth of microbes, especially dermatophytes, present on the surface of the scalp and within the epidermis thereof may be suppressed, thus improving the health of the scalp and preventing hair loss.

Such hexose is preferably contained in an amount of 7 to 28 wt % in the complex buffer solution. If the amount of hexose is low, it is difficult to obtain desired effects. On the other hand, if the amount thereof exceeds the above upper limit, the effects are not further increased despite excessive use thereof and viscosity may increase, undesirably causing a poor sensation of use due to an oily feeling, stickiness, or heavy feeling upon use.

The polyhydric alcohol is in a solid phase at room temperature (25° C.), and may be typically obtained by reducing a carbonyl group of a saccharide, and preferably includes at least one selected from the group consisting of maltitol, sorbitol, ribitol, mannitol, arabitol, galactitol, xylitol, erythritol, and inositol.

More preferably useful are sorbitol and/or maltitol, which do not exhibit stickiness and stiffness and thus have a good sensation of use when being contained in the cosmetic composition. These are absorbed into the scalp together with hexose and are thus used as an energy source of cells, thereby maintaining the healthy hair, and are also involved in the biosynthesis of nucleic acid, whereby the lipid metabolism is improved thanks to the anti-ketone effect, thus aiding the recovery of cells in which aging is progressed in the epidermis of the scalp, ultimately preventing hair loss.

The polyhydric alcohol may be contained in an amount of 2 to 8 wt % in the buffer solution. If the amount thereof is less than 2 wt %, the desired effects cannot be obtained. On the other hand, if the amount thereof exceeds 8 wt %, viscosity may increase, and the cosmetic composition for improving the health of the scalp and preventing hair loss according to the present invention, containing the same, may cause stickiness or a feeling of heavy hair when applied on the hair, undesirably deteriorating the feeling of use.

The cosmetic composition for improving the health of the scalp according to the present invention may further include an additional ingredient depending on the aspect and method of use thereof, and examples of the additional ingredient may include a disinfectant, a preservative, a binder, a thickener, a fixer, an excipient, a colorant, a stabilizer, a pH controller, a buffering agent, a tonicity agent, a solvent, an antioxidant, a UV inhibitor, a crystal precipitation inhibitor, an antifoaming agent, and a characteristic improver.

The disinfectant is not particularly limited, and may be appropriately selected depending on the end use. Examples thereof may include cationic surfactants such as benzalkonium chloride, benzethonium chloride, and cetylpyridinium chloride. The preservative is not particularly limited, and may be appropriately selected depending on the end use. Examples thereof may include p-hydroxybenzoate esters, chlorobutanol, and cresol.

The binder, the thickener and the fixer are not particularly limited, and may be appropriately selected depending on the end use, and examples thereof may include starch, dextrin, cellulose, methyl cellulose, ethyl cellulose, carboxy methyl cellulose, hydroxyl ethyl cellulose, hydroxyl propyl cellulose, hydroxypropyl methyl cellulose, carboxy methyl starch, pullulan, sodium alginate, ammonium alginate, propylene glycol ester alginate, guar gum, locust bean gum, Arabia rubber, xanthan gum, gelatin, casein, polyvinyl alcohol, polyethylene oxide, polyethylene glycol, ethylene/propylene block polymer, sodium polyacrylate, and polyvinyl pyrrolidone, which may be used alone or in combinations of two or more thereof.

Furthermore, the binder is not particularly limited, and may be appropriately selected depending on the end use, and examples thereof may include water, ethanol, propanol, simple syrup, a glucose solution, a starch solution, a gelatin solution, carboxymethyl cellulose, hydroxyl propyl cellulose, hydroxypropyl starch, methyl cellulose, ethyl cellulose, shellac, calcium phosphate, and polyvinyl pyrrolidone.

The colorant is not particularly limited, and may be appropriately selected depending on the end use, and examples thereof may include titanium oxide and iron oxide.

The stabilizer is not particularly limited, and may be appropriately selected depending on the end use, and examples thereof may include tragacanth, gum arabic, gelatin, sodium pyrosulfite, EDTA, thioglycolic acid, and thiolactic acid.

The pH controller and the buffering agent are not particularly limited, and may be appropriately selected depending on the end use, and examples thereof may include sodium citrate, sodium acetate, and sodium phosphate.

The tonicity agent is not particularly limited, and may be appropriately selected depending on the end use, and examples thereof may include sodium chloride and glucose.

Also, a fragrance additive may be added to exhibit effects such as aromatherapy to relieve the stress of modern people and a scalp stabilization effect, and may include, but is not limited to, at least one selected from the group consisting of menthol, lavandin oil, bisabolol, sorbitol, and rosehip oil.

In an exemplary embodiment, the menthol aids mental stability and blood circulation in the scalp and functions to cool the scalp heat, and the lavandin oil functions to promote peace of mind and body and is effective at relieving skin troubles and at regenerating the damaged scalp tissue.

The fragrance additive is preferably used in an amount of 0.01 to 0.03 parts by weight based on 100 parts by weight of the composition. If the amount thereof is less than 0.01 parts by weight, the aromatherapy effect or skin stabilization effect may become poor. On the other hand, if the amount thereof exceeds 0.03 parts by weight, scalp irritation may occur.

The cosmetic composition of the present invention may be prepared in any formulation that is typical in the art, and is preferably provided in the form of any formulation selected from the group consisting of a solution, a suspension, an emulsion, a paste, a gel, a cream, a lotion, a powder, an oil, a soap, a cleaning foam, a shampoo, a rinse, a treatment, a wax, and a spray, but is not limited thereto.

More preferably, when the formulation of the cosmetic composition for improving the health of the scalp according to the present invention is a paste, a cream or gel, an animal oil, a vegetable oil, wax, paraffin, starch, tragacanth, cellulose derivatives, polyethylene glycol, silicone, bentonite, silica, talc or zinc oxide may be further included. When the formulation thereof is a powder or a spray, lactose, talc, silica, aluminum hydroxide, calcium silicate, or a polyamide powder may be further included. Particularly in the case of a spray formulation, a propellant such as chlorofluorohydrocarbon, propane/butane or dimethyl ether may be further included.

When the formulation of the cosmetic composition of the present invention is a solution or an emulsion, a solvent, a solvating agent or an emulsifier may be further included, and examples thereof may include water, ethanol, isopropanol, ethyl carbonate, ethyl acetate, benzyl alcohol, benzyl benzoate, propylene glycol, 1,3-butylglycol oil, glycerol aliphatic ester, polyethylene glycol or sorbitan fatty acid ester.

When the formulation of the cosmetic composition of the present invention is a suspension, a liquid diluent such as water, ethanol or propylene glycol, a suspension agent such as ethoxylated isostearyl alcohol, polyoxyethylene sorbitol ester and polyoxyethylene sorbitan ester, microcrystalline cellulose, aluminum metahydroxide, bentonite, agar or tragacanth may be further included.

Also, the cosmetic composition for improving the health of the scalp according to the present invention may be applied to any body region including not only the scalp but also a body portion that needs hair growth. For example, it may be used for portions with damaged hair due to traumatic scars, or for improving the appearance of a wide forehead or an M-shaped forehead, eyelashes, eyebrows, and atrichia for simple cosmetic effects.

Examples of the hair to which the cosmetic composition of the present invention may be applied may include any type of body hair, such as head hair, eyebrows, eyelashes, pubic hair, armpit hair, chest hair, nose hair, and leg hair.

In addition, another embodiment of the present invention addresses a method of preparing the cosmetic composition for improving the health of the scalp and preventing hair loss, comprising: preparing an oligopeptide configured to include glycine as an N-terminus amino acid and to have a length of 10 to 15 amino acids; mixing the oligopeptide with an insulin-like growth factor-1 and thymosin-β4, thus preparing a mixture; and adding the mixture with any one selected from among an oil, a surfactant, a moisturizer, a conditioning agent, an antioxidant, a thickener, a binder, a pH controller, a buffering agent, a colorant and a fragrance, thus obtaining the cosmetic composition.

Specifically, the preparing the oligopeptide is not particularly limited so long as it is a typical peptide synthesis process, and preferably useful is a solid peptide synthesis process using a solid polymer support, whereby the α-amino group of the oligopeptide thus prepared may be protected by an acid- or base-sensitive functional group. The protecting group of the amino acid has to be stable under the peptide condensation conditions, and should be able to be easily removed, without breakage of an extending peptide chain or without racemization of any chiral center contained therein.

Examples of the protecting group may include 9-fluorenylmethyloxycarbonyl (Fmoc), t-butoxy carbonyl (Boc), benzyloxy carbonyl (Cbz), biphenyl isopropyl-oxy carbonyl, t-amyloxy carbonyl, isobornyl oxy carbonyl, (α,α)-dimethyl-3,5-dimethoxybenzyloxy carbonyl, o-nitrophenylsulfenyl, and 2-cyano-t-butyloxy carbonyl, and other protecting groups known in the art for this purpose may also be used within the scope of the present invention.

Particularly useful as the protecting group of the amino acid used for synthesis of the oligopeptide according to the present invention is 9-fluorenylmethyloxycarbonyl (Fmoc).

In particular, the protecting group of the amino acid residue used for the synthesis of the oligopeptide according to the present invention is t-butyl (t-Bu) for N-methyl glutamic acid; t-butoxycarbonyl (Boc) for lysine; 7t-butyl (t-Bu) for serine; t-butyl (t-Bu) for threonine and allo-threonine; and trityl (Trt) for cysteine, but the present invention is not limited thereto.

In the solid peptide synthesis process, a C-terminus amino acid may be attached to an appropriate solid support or resin. The solid support suitable for the above synthesis process is preferably a material that is inert for reagents and reaction conditions of stepwise condensation-deprotection reactions and is insoluble in a medium used, and may be exemplified by a rink amide or a rink amide 4-methylbenzylhydrylamine (MBHA) resin.

Particularly, the solid support suitable for the C-terminus amide peptide may be a rink amide 4-methylbenzylhydrylamine resin available from Novabiochem Corporation.

The C-terminus amide may be condensed (linked or coupled) to the resin or solid support by activating carboxylic acid on N,N′-dicyclohexyl carbodiimide (DCC), N,N′-diisopropyl carbodiimide (DIC), [O-(7-azabenzotriazol-1-yl)-1,1,3,3-tetramethyluronium hexafluorophosphate] (HATU) or O-benzotriazol-1-yl-N,N,N′,N′-tetramethyluronium hexafluorophosphate (HBTU) at a temperature of 10 to 50° C., and preferably 30° C., for 1 to 24 hr using a solvent such as dichloromethane, N-methylpyrrolidone (NMP) or DMF in the presence or absence of 4-dimethylaminopyridine (DMAP), 1-hydroxybenzotriazole (HOBt), N-methylmorpholine (NMM), benzotriazol-1-yloxy-(BOP) or bis(2-oxo-3-oxazolidinyl)phosphine chloride (BOPCI).

When the solid support is a rink amide 4-methylbenzylhydrylamine resin, the preferable protecting group, namely the Fmoc functional group is cleaved using an excess of a secondary amine solution and preferably a 20% piperidine DMF solution before condensation into the C-terminus amino acid. Examples of the reagent used to condense the desired amino acid to the deprotected 4-(2′,4′-dimethoxyphenyl-Fmoc-aminomethyl)phenoxyacetamidoethyl resin may include condensation reagents in the DMF solvent for the appropriately protected amino acid, such as N-methylmorpholine (NMM), 1-hydroxybenzotriazole (HOBt) and O-(7-azabenzotriazol-1-yl)-1,1,3,3-tetramethyluronium hexafluorophosphate] (HATU), O-benzotriazol-1-yl-N,N,N′,N′-tetramethyluronium hexafluorophosphate (HBTU), N,N′-dicyclohexyl carbodiimide (DCC), and N,N′-diisopropyl carbodiimide (DIC).

The continuous amino acid condensation process of the present invention may be performed using an automatic peptide synthesizer that is widely known in the related art, or may be manually and directly conducted. As for the preferred synthesis conditions, the Fmoc-protected glycine monomer is deprotected with a secondary amine solution, and preferably piperidine, sufficiently washed with an excess of solvent, added with individual protected amino acids to be condensed in an excessive 3- to 7-fold molar amount, and reacted in the DMF solvent, thereby yielding the oligopeptide in which the N-terminus amino acid is glycine.

In the final step of synthesis of the oligopeptide using the solid resin according to the present invention, the oligopeptide to be obtained is removed from the resin through continuous processing or a single operation, and the protecting group for protecting each amino acid residue may be deprotected. For removal of the oligopeptide from the resin and deprotection of the protecting group from the residue, a cleavage cocktail for cleaving the resin-peptide bonding, for example, a dichloromethane-mixed cocktail solution comprising trifluoroacetic acid (TFA), triisopropylsilane (TIS), thioanisole, water or ethanedithiol (EDT) may be used.

The mixed solution thus obtained is treated with an excess of a refrigerated diethylether solvent, whereby a precipitate may be formed. The precipitate thus obtained is centrifuged and completely deposited, after which excess trifluoroacetic acid, triisopropylsilane, thioanisole, water and ethanedithiol are primarily removed, and these procedures are repeated two times or more, thereby obtaining a solidified precipitate.

Here, the completely deprotected oligopeptide salt may be separated and purified using a solvent mixture comprising water and acetonitrile through reverse-phase high-performance liquid chromatography (HPLC). The oligopeptide solution thus purified is completely concentrated and dried through lyophilization, thereby yielding a solid oligopeptide.

In the above preparation method, the mixing the solid oligopeptide in which the N-terminus amino acid is glycine and which has a length of 10 to 15 amino acids with the insulin-like growth factor-1 and thymosin-β4 is performed in a manner in which a surfactant is added to a water phase at 30 to 90° C., and the water phase containing the surfactant is mixed with an organic phase to give a solvent, in which the oligopeptide, insulin-like growth factor-1 and thymosin-β4 are then included, followed by homogenization at a high pressure of 50 to 2000 bar, thus obtaining an emulsion in which the oligopeptide, insulin-like growth factor-1 and thymosin-β4 are incorporated by mechanical stress in the carrier, which is then cooled, resulting in a mixture.

In the above mixing step, a complex buffer solution including hexose, a polyhydric alcohol and an electrolyte compound may be further included. The other components, in addition to the essential components of the mixture such as the oligopeptide, insulin-like growth factor-1, thymosin-β4 and complex buffer solution, may be appropriately chosen and combined by those skilled in the art depending on the end use.

Preferably, any one selected from the group consisting of an oil, a surfactant, a moisturizer, a conditioning agent, an antioxidant, a thickener, a binder, a pH controller, a buffering agent, a colorant and a fragrance may be further added, followed by the preparation of the cosmetic composition, ultimately resulting in a cosmetic composition for improving the health of the scalp and preventing hair loss according to the present invention.

A better understanding of the present invention may be obtained through the following Examples, which are merely set forth to illustrate, but are not construed as limiting the scope of the present invention, and various modifications thereof may be performed within the scope of the present invention by those skilled in the art.

### Preparation Example 1

Preparation of Oligopeptide

A 2-chlorotrityl chloride resin available from Novabiochem Corp. (loaded in 0.6 mmol per g) was placed in an automatic peptide synthesizer. The resin was solvated with 15 ml of DMF, sufficiently swelled for 5 min, added with 15 ml of a 20% (w/v) piperidine DMF solution, and shaken, after which the piperidine DMF solution was removed, and washing was performed with 15 ml of a DMF solvent and 15 ml of an MC solvent and then further conducted with 15 ml of an MC solvent.

0.5 mmol of Fmoc-gly-OH, 0.5 mmol of 1-O-benzotriazole-N,N,N′,N′-tetramethyluronium hexafluorophosphate (HBTU), and 0.5 mmol of N,N-diisopropylethylamine (DIEA) were dissolved in 20 ml of anhydrous DMF and activated for 4 min. Thereafter, the activated solution was mixed with a resin and then subjected to a coupling reaction for 30 min. The resin was rinsed with 20 ml of MC and 20 ml of DMF for 40 sec, and the Fmoc protecting group was removed using 20% piperidine dissolved in DMF.

Thereafter, the resin was washed again, and depending on the preset amino acid sequence, Fmoc-protected amino acids (Fmoc-Gln(Trt), Fmoc-Gln(Trt), Fmoc-Lys(Boc), Fmoc-Arg(Pbf), Fmoc-Ser(tBu), Fmoc-Asp(OtBu), Fmoc-Arg(Pbf), Fmoc-Leu, Fmoc-Asn(Trt), Fmoc-Leu, Fmoc-Ser(tBu), Fmoc-Arg(Pbf), in that order) were sequentially added and reacted. After the completion of synthesis of the entire amino acid sequence, unreacted amine was shaken for 10 min using an acetic anhydride and rutidine-containing DMF.

In order to protect the unreacted amine with 2-(9H-fluoren-9-yl)ethylcarbamate, it was reacted with 2-(9H-fluoren-9-yl)ethylcarbamate-O-succinimide for 10 min, and the resin was rinsed for 40 sec with 20 ml of MC and 20 ml of DMF.

A leaving solution [2% TFA (Trifluoroacetic acid), 2% triisopropylsilane] was added and reacted for 2 hr with occasional shaking at room temperature, and the resin was filtered, precipitated with cold ether, and dried, thus obtaining an oligopeptide.

The obtained oligopeptide was purified through reverse-phase HPLC using a 10% to 90% acetonitrile/water concentration gradient solvent system by means of a C-18 column for 100 min at a flow rate of 25 to 35 ml/min.

### Test Example 1

Stability of Oligopeptide

In order to evaluate the stability of the oligopeptide prepared in Preparation Example 1 when applied to humans, irritation of the skin and scalp due to cytotoxicity was measured using a HaCaT cell line as human keratinocytes.

The HaCaT cell line used therefor was incubated at 37° C. in a 95% CO₂ incubator. When the extent of incubation arrived at 85 to 90% of the area of a culture vessel, the cells were detached with trypsin, counted, and subjected to subculture at 5×10³ cells/cm².

For cell culture, a Dulbecco's Modified Eagle Medium (DMEM, GIBCO, Cat. No. 11995-065, USA), containing 10% fetal bovine serum (FBS, GIBCO, Cat. No., 26140-079, USA), 100 U/ml penicillin, and 100 μg/ml streptomycin, was used. For subculture, a 75 T-flask (NUNC, Cat. No. 156499, Denmark) was used, and the cytotoxicity test was carried out using a 24-well plate (NUNC, Cat. No. 142475, Denmark).

The human keratinocytes were counted at 5×10³ cells/well each and then aliquoted into the 24-well plate using a hemocytometer. These cells were incubated in DMEM containing 10% FBS for 48 hr. When the extent of incubation arrived at 40 to 50% of the surface area of the culture vessel, the medium was replaced with FBS-free DMEM containing each of 50 ppm oligopeptide and 100 ppm oligopeptide, and culture was further conducted for 24 hr. Thereafter, 50 μl of a 3-(4,5-dimethylthiazol-2-yl)-2,5-diphenyl tetrazolium bromide (MTT, Sigma M5655, USA) solution (2.5 mg/ml) was added and culture was further performed for 3 hr. Then, the cell culture broth was discarded, 200 μl of dimethyl sulfoxide (DMSO, Sigma D2650, USA) was added to each well and shaken, 100 μl thereof was transferred into a 96-well plate, and the absorbance thereof was measured at 570 nm using an Enzyme-Linked Immunosorbent Assay (ELISA).

The cytotoxicity was represented as a percentage based on the absorbance intensity of a control using pure water. The results are shown in Table 1 below.

TABLE 1

| | Cytotoxicity (%) |
|---|---|
| Control (pure water) | 100 |
| 50 ppm Oligopeptide | 93 |
| 100 ppm Oligopeptide | 81 |

As is apparent from the results of Table 1, when initial 5×10³ cells per well were incubated to cover 40 to 50% of the surface area of the culture vessel, the groups added with 50 ppm oligopeptide and 100 ppm oligopeptide exhibited no cytotoxicity compared to the control. This means that skin and scalp irritation is not expected to occur due to cytotoxicity even upon the long-term use of the oligopeptide obtained in Preparation Example 1.

### Preparation Example 2

Preparation of Cosmetic Composition

A cosmetic composition was prepared by mixing the oligopeptide prepared in Preparation Example 1, an insulin-like growth factor-1, thymosin-β4, a complex buffer solution comprising hexose, a polyhydric alcohol and an electrolyte compound, and additional components in the amounts shown in Table 2 below.

Specifically, the following additional components were mixed, and warmed to 80° C. to thus give a single-phase form, which was then mixed with the oligopeptide, the insulin-like growth factor-1, thymosin-β4 and the complex buffer solution at 55° C., passed three times through a high-pressure emulsifying machine at 1000 bar, and then cooled, thus yielding the cosmetic compositions of Examples 1 to 3 and Comparative Examples 1 to 4.

TABLE 2

| | | | C. Ex. 1 | C. Ex. 2 | C. Ex. 3 | C. Ex. 4 | Ex. 1 | Ex. 2 | Ex. 3 |
|---|---|---|---|---|---|---|---|---|---|
| Oligopeptide | | | 0.76 | 2.62 | — | 1.86 | 0.81 | 1.86 | 2.45 |
| Insulin-like growth | | | | | | | | | |
| factor-1 (IGF-1) | | | 10.0 | 10.0 | 10.0 | 10.0 | 10.0 | 10.0 | 10.0 |
| Thymosin-β4 | | | 4.98 | 4.98 | 4.98 | 4.98 | 1.73 | 4.98 | 6.70 |
| Complex | Hexose | D- | | | | | | | |
| buffer | | fructose | 0.14 | 0.14 | 0.14 | — | 0.14 | 0.14 | 0.14 |
| solution | | D- | 0.08 | 0.08 | 0.08 | — | 0.08 | 0.08 | 0.08 |
| | | glucose | | | | | | | |
| | Polyhydric | Sorbitol | 0.06 | 0.06 | 0.06 | — | 0.06 | 0.06 | 0.06 |
| | alcohol | | | | | | | | |

| | | | | | | | | |
|---|---|---|---|---|---|---|---|---|
| | Electrolyte compound | 0.0035 | 0.0035 | 0.0035 | — | 0.0035 | 0.0035 | 0.0035 |
| | Water (Remainder) | 0.7165 | 0.7165 | 0.7165 | 1.00 | 0.7165 | 0.7165 | 0.7165 |
| Additional | Carbomer | 0.016 | 0.016 | 0.016 | 0.016 | 0.016 | 0.016 | 0.016 |
| component | Triethanolamine | 0.016 | 0.016 | 0.016 | 0.016 | 0.016 | 0.016 | 0.016 |
| | Phenoxyethanol | 0.3 | 0.3 | 0.3 | 0.3 | 0.3 | 0.3 | 0.3 |
| | Menthol | 0.01 | 0.01 | 0.01 | 0.01 | 0.01 | 0.01 | 0.01 |
| | PEG-60 hydrogenated | | | | | | | |
| | castor oil | 0.03 | 0.03 | 0.03 | 0.03 | 0.03 | 0.03 | 0.03 |
| | Purified water | Remainder | Remainder | Remainder | Remainder | Remainder | Remainder | Remainder |

(unit: based on the total weight 100 g)

### Test Example 2

Evaluation of Percutaneous Absorption Rate of Cosmetic Composition

In order to evaluate the hair-loss prevention effect of the cosmetic composition of Preparation Example 2, the percutaneous absorption test was performed. The percutaneous absorption was conducted in a manner in which the skin of a hairless guinea pig was sectioned to an area of 1 cm², after which each sample was weighed to 0.5 g and treated so as not to be contaminated. After 30 min, testing was performed four times through a tape-stripping process. Based on the amount of the amino group on the skin surface, the concentration was analyzed after solvent treatment using HPLC. The results are shown in Table 3 below.

Comparative Example 5 was performed in the same manner as in Example 2 of Preparation Example 2, with the exception that mixing was implemented using a shaker in lieu of the high-pressure emulsifying machine.

TABLE 3

| | C. Ex. 1 | C. Ex. 2 | C. Ex. 3 | C. Ex. 4 | C. Ex. 5 | Ex.1 | Ex.2 | Ex.3 |
|---|---|---|---|---|---|---|---|---|
| 1 | 61.2 | 48.5 | 73.1 | 67.1 | 75.8 | 51.6 | 49.5 | 47.9 |
| 2 | 37.1 | 33.1 | 23.1 | 37.2 | 23.0 | 30.8 | 31.2 | 33.7 |
| 3 | 11.0 | 17.7 | 6.7 | 9.3 | 7.0 | 17.9 | 19.0 | 20.1 |
| 4 | 3.4 | 9.9 | 1.1 | 2.7 | 0.0 | 15.1 | 16.7 | 17.9 |

As is apparent from the test results, in the formulations of Comparative Examples 1 and 3 to 5, the oligopeptide of the present invention and the growth factor were mainly present on the skin surface and thus skin penetration did not occur. Particularly in Comparative Example 5, little skin penetration occurred.

In contrast, in the formulations of Examples 1 to 3 and Comparative Example 2, penetration into the skin occurred to the greatest extent.

Specifically, the cosmetic composition obtained by mixing the oligopeptide of the present invention, the insulin-like growth factor-1, and thymosine-β4 in preferred amounts and then incorporating the mixture using the carrier of the present invention exhibited significantly improved skin absorption capability.

### Test Example 3

Sensory Evaluation of Cosmetic Composition (Hair-Loss Prevention Effect)

In order to evaluate the hair-loss prevention effect of the cosmetic composition of Preparation Example 2, 50 adult male and female persons each were tested for 3 months using the cosmetic compositions of Examples 1 to 3 and Comparative Examples 1 to 5, and the effects thereof were measured.

TABLE 4

| | | | | Significant |
|---|---|---|---|---|
| | Severe hair | No change | Slight prevention of | prevention of |
| | loss | after use | hair loss | hair loss |
| C. Ex. 1 | 8/50 | 12/50 | 21/50 | 9/50 |
| C. Ex. 2 | 1/50 | 3/50 | 20/50 | 26/50 |
| C. Ex. 3 | 12/50 | 12/50 | 23/50 | 3/50 |
| C. Ex. 4 | 10/50 | 13/50 | 25/50 | 2/50 |
| C. Ex. 5 | 9/50 | 22/50 | 14/50 | 5/50 |
| Ex. 1 | 1/50 | 5/50 | 9/50 | 35/50 |
| Ex. 2 | 0/50 | 6/50 | 7/50 | 37/50 |
| Ex. 3 | 0/50 | 2/50 | 10/50 | 38/50 |

As is apparent from the results of Table 4, the hair-loss prevention effect was higher in Examples 1 to 3 than in Comparative Examples 1 to 5.

In Comparative Example 2 containing the oligopeptide in the highest amount, the hair-loss prevention effect was somewhat low compared to Examples 1 to 3, and the improvement in the health of the scalp and the prevention of hair loss were relatively superior but the feeling of use was poor due to the stiff or heavy sensation in use.

### Test Example 4

Sensory Evaluation of Cosmetic Composition Containing Oligopeptide (Hair-Loss Prevention Effect)

In order to evaluate the hair-loss prevention effect, 50 adult male and female persons each were tested for 3 months using the cosmetic composition of Example 2 and the cosmetic compositions of Examples 4 and 5 prepared in the same manner as in Example 2 containing the oligopeptide having the sequence of Gly-His-Lys-Gly-Gin-Leu-Tyr-Val-Gln-Len and the oligopeptide having the sequence of Gly-His-Lys-Lys-Gly-His-Lys-Glu-Gln-Arg-Tyr-Val-Gln-Leu-Tyr, respectively, and the effects thereof were measured.

TABLE 5

| | | | Slight | |
|---|---|---|---|---|
| | Severe hair | No change | prevention of | Significant prevention of |
| | loss | after use | hair loss | hair loss |
| Ex. 2 | 0/50 | 2/50 | 10/50 | 38/50 |
| Ex. 4 | 0/50 | 3/50 | 11/50 | 36/50 |
| Ex. 5 | 0/50 | 1/50 | 10/50 | 39/50 |

As is apparent from the results of Table 5, the effect of preventing hair loss in Examples 4 and 5 was similar to that of Example 2.

Therefore, the cosmetic composition for improving the health of the scalp according to the present invention can control sebum secretion of the scalp to thus create an environment suitable for hair growth, and can also activate growth factors that are effective at preventing hair loss by promoting hair growth through the improvement of blood circulation in the scalp and by strengthening hair through hair follicle activity. Moreover, when this composition is applied on the scalp, no irritation occurs, and it can penetrate deeply at a high concentration, thus aiding in preventing hair loss through improving the health of the scalp.

Although the embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A cosmetic composition for improving health of a scalp, comprising: an insulin-like growth factor-1, thymosin-β4 and an oligopeptide, wherein the oligopeptide is configured to include glycine as an N-terminus amino acid and to have a length of 10 to 15 amino acids.

2. The cosmetic composition of claim 1, comprising 100 parts by weight of the insulin-like growth factor-1, 17 to 67 parts by weight of the thymosin-β4 and 8 to 25 parts by weight of the oligopeptide.

3. The cosmetic composition of claim 1, wherein the insulin-like growth factor-1, the thymosin-β4 and the oligopeptide are incorporated in a carrier and stabilized.

4. The cosmetic composition of claim 3, wherein the carrier is a macromolecule, a microassembly, a microparticle, a microsphere, a nanosphere, a liposome, an emulsion or a combination thereof.

5. The cosmetic composition of claim 1, further comprising a complex buffer solution including hexose, a polyhydric alcohol and an electrolyte compound to maintain homeostasis of an acid and a base inside and outside a scalp cell.

6. The cosmetic composition of claim 5, wherein the complex buffer solution includes 7 to 28 wt % of the hexose, 2 to 8 wt % of the polyhydric alcohol, 0.23 to 1.559 wt % of the electrolyte compound and a remainder of water, and a sum of the hexose, the polyhydric alcohol, the electrolyte and the water does not exceed 100 wt %.

7. The cosmetic composition of claim 1, wherein the cosmetic composition is provided in any formulation selected from the group consisting of a solution, a suspension, an emulsion, a paste, a gel, a cream, a lotion, a powder, an oil, a soap, a cleansing foam, a shampoo, a rinse, a treatment, a wax, and a spray.

8. A method of preparing a cosmetic composition for improving health of a scalp, comprising:
preparing an oligopeptide configured to include glycine as an N-terminus amino acid and to have a length of 10 to 15 amino acids;
mixing the oligopeptide with an insulin-like growth factor-1 and thymosin-β4, thus preparing a mixture; and
adding the mixture with any additive selected from the group consisting of an oil, a surfactant, a moisturizer, a conditioning agent, an antioxidant, a thickener, a binder, a pH controller, a buffering agent, a colorant and a fragrance, thus obtaining the cosmetic composition.

9. The method of claim 8, wherein in the mixing, the oligopeptide, the insulin-like growth factor-1 and the thymosin-β4 are included in a solvent obtained by mixing an organic phase, a water phase and a surfactant, homogenized at a high pressure and then cooled.

10. The method of claim 8, wherein in the mixing, a complex buffer solution including hexose, a polyhydric alcohol and an electrolyte compound is further included.
